(19) United States
(12) Patent Application Publication
(10) Pub. No.: US 2025/0375276 A1
(43) Pub. Date: Dec. 11, 2025

(54) ORAL IRRIGATOR AND NOZZLE THEREOF

(71) Applicant: GUANGZHOU STARS PULSE CO.,LTD., GUANGZHOU (CN)

(72) Inventor: YUANJIAN LIANG, GUANGZHOU (CN)

(21) Appl. No.: 18/892,742

(22) Filed: Sep. 23, 2024

(30) Foreign Application Priority Data
Jun. 7, 2024 (CN) 202421309874.0

(51) Int. Cl. A61C 17/02; A61M 3/02

(52) U.S. Cl. CPC A61C 17/02; A61M 3/0279

(57) ABSTRACT

A nozzle and an oral irrigator include a squirting tube. The squirting tube includes an inlet, an outlet, and a flow channel. The squirting tube includes a first bending position located between a first bending section, and a second bending section and a second bending position disposed between the second bending position and the outlet. An extension direction of the first bending section is different from an extension direction of the main flow section and an extension direction of the second bending section. The flow channel in the main flow section defines a first central axis. The outlet is located on a first side of the first central axis. The first bending section bends and extends from the main flow section to a second side, opposite to the first side, of the first central axis.

### TECHNICAL FIELD

The present disclosure relates to a technical field of oral cleaning, and in particular to an oral irrigator and a nozzle thereof.

### BACKGROUND

An oral irrigator is an electronic device configured to clean an oral cavity and generally comprises a main body and a nozzle. The nozzle comprises a squirting tube and a squirting head. Currently, most oral irrigators use a standard hot-bent nozzle to irrigate teeth. However, it is easy to irrigate teeth at a front of the oral cavity by the standard hot-bent nozzle, but it difficult to clean teeth deep in the oral cavity by the standard hot-bent nozzle. For example, a user need to open his/her mouth wide to clean the teeth deep in the oral cavity, but it is still difficult to clean residue in gaps between the teeth deep in the oral cavity, resulting in poor cleaning effect.

### SUMMARY

Embodiments of the present disclosure provide a nozzle of an oral irrigator and the oral irrigator.

In a first aspect, the nozzle of the present disclosure comprises a squirting tube. The squirting tube comprises an inlet, an outlet, and a flow channel communicating the inlet and the outlet. The squirting tube at least comprises a first bending position and a second bending position, so as to form a main flow section, a first bending section, and a second bending section in a direction from the inlet to the outlet. The main flow section, the first bending section, and the second bending section are sequentially connected. The first bending section is disposed between the first bending position and the second bending position. The second bending section is disposed between the second bending position and the outlet. An extension direction of the first bending section is different from an extension direction of the main flow section, and the extension direction of the first bending section is different from an extension direction of the second bending section. A section of the flow channel in the main flow section defines a first central axis. The outlet is located on a first side of the first central axis. The first bending section bends and extends from the main flow section to a second side of the first central axis. The first side of the first central axis is opposite to the second side of the first central axis.

In a second aspect, the oral irrigator of the present disclosure comprises a nozzle and a main body. The nozzle comprises a squirting tube. The squirting tube comprises an inlet, an outlet, and a flow channel communicating the inlet and the outlet. The squirting tube at least comprises a first bending position and a second bending position, so as to form a main flow section, a first bending section, and a second bending section in a direction from the inlet to the outlet. The main flow section, the first bending section, and the second bending section are sequentially connected. The first bending section is disposed between the first bending position and the second bending position. The second bending section is disposed between the second bending position and the outlet. An extension direction of the first bending section is different from an extension direction of the main flow section, and the extension direction of the first bending section is different from an extension direction of the second bending section. A section of the flow channel in the main flow section defines a first central axis. The outlet is located on a first side of the first central axis. The first bending section bends and extends from the main flow section to a second side of the first central axis. The first side of the first central axis is opposite to the second side of the first central axis. The main body is connected to the nozzle.

The nozzle comprises the first bending section and the second bending section. The first bending section bends and extends from the main flow section toward the second side of the first central axis of the main flow section, and the second bending section bends and extends from the first bending section. When the nozzle is inserted into an oral cavity and cleans a deep part of the oral cavity to be cleaned, the first bending section is closer to lips and teeth at a front of the oral cavity than the second bending section. The second bending section is deeper into the oral cavity and closer to teeth at the deep part of the oral cavity than the first bending section. In addition, since the first bending section bends and extends toward the second side of the first central axis of the main flow section, the first bending section and the main flow section jointly define a first space on the second side of the first central axis. The first space provides a space for the lips and the teeth at the front of the oral cavity, so the first bending section does not contact the lips and the teeth at the front of the oral cavity when the nozzle cleans the deep part of the oral cavity to be cleaned. On the one hand, the first space prevents the nozzle from hitting the lips and the teeth at the front of the oral cavity to avoid damage to the human body. On the other hand, the first space increase an amplitude of the nozzle turning and shifting in the oral cavity and expands a cleaning area of the nozzle. Moreover, by providing the first bending section and the second bending section, a user does not need to open his mouth wide when putting the nozzle into the mouth.

Additional aspects and characteristics of the embodiments of the present disclosure are given in part in the following description and in part are obvious from the following description, or are learned through practice of the embodiments of the present disclosure.

### BRIEF DESCRIPTION OF DRAWINGS

The above aspects and/or additional aspects and characteristics of the present disclosure may be apparent and easily understood from the description of the embodiments in conjunction with the following drawings.

FIG. 1 is a perspective schematic diagram of a nozzle according to a first embodiment of the present disclosure.

FIG. 2 is a cross-sectional schematic diagram of the nozzle shown in FIG. 1.

FIG. 3 is a perspective schematic diagram of the nozzle shown in an assembled state according to a second embodiment of the present disclosure.

FIG. 4 is an exploded perspective schematic diagram of the nozzle shown in FIG. 3.

FIG. 5 is a cross-sectional schematic diagram of the nozzle shown in FIG. 3.

FIG. 6 is a perspective schematic diagram of the nozzle shown in an assembled state according to a third embodiment of the present disclosure.

FIG. 7 is an exploded perspective schematic diagram of the nozzle shown in FIG. 6.

FIG. 8 is a cross-sectional schematic diagram of the nozzle shown in FIG. 6.

FIG. 9 is a partial perspective schematic diagram of the nozzle shown in FIG. 6.

FIG. 10 is a perspective schematic diagram of the nozzle shown in an assembled state according to a fourth embodiment of the present disclosure.

FIG. 11 is an exploded perspective schematic diagram of the nozzle shown in FIG. 10.

FIG. 12 is a cross-sectional schematic diagram of the nozzle shown in FIG. 11.

FIG. 13 is a perspective schematic diagram of an oral irrigator according to one embodiment of the present disclosure.

Reference numbers in the drawings: oral irrigator 1000; nozzle 100; main body 300; squirting tube 10; inlet 11; outlet 12; flow channel 13; first flow channel 131; second flow channel 133; first bending position 14; second bending position 15; main flow section 16; first side 161; second side 163; first sub-section 165; second sub-section 167; first bending section 17; first connecting port 171; second connecting port 173; second bending section 18; outer wall 19; limiting protrusion 191; squirting head 30; channel 31; liquid inlet 33; liquid outlet 35; peripheral wall 351; notch 3511; inner wall 37; groove 371; bristles 39.

### DETAILED DESCRIPTION

Embodiments of the present disclosure are described in detail below, and examples of the embodiments are shown in the accompanying drawings. The same or similar reference numerals represent the same or similar elements or elements having the same or similar functions throughout the present disclosure.

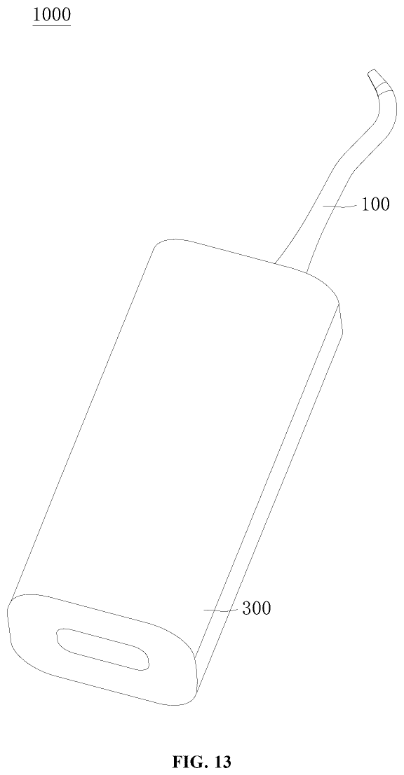

An oral irrigator 1000 (shown in FIG. 13 of the embodiments of the present disclosure comprises a nozzle 100 and a main body 300 (shown in FIG. 13). The nozzle 100 is connected to the main body 300.

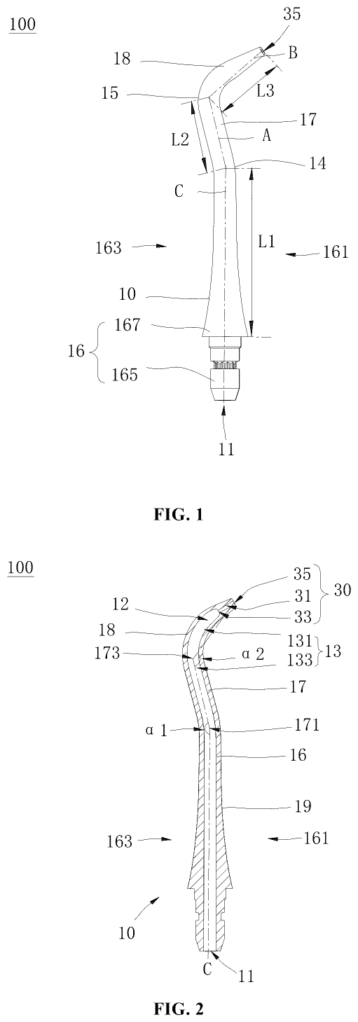

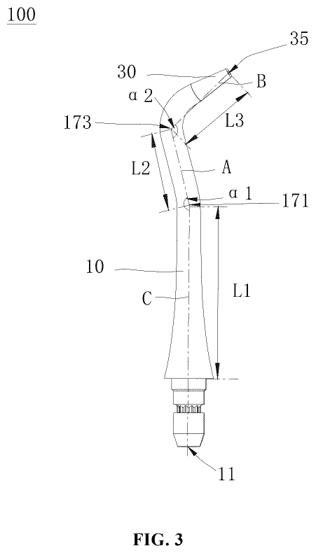

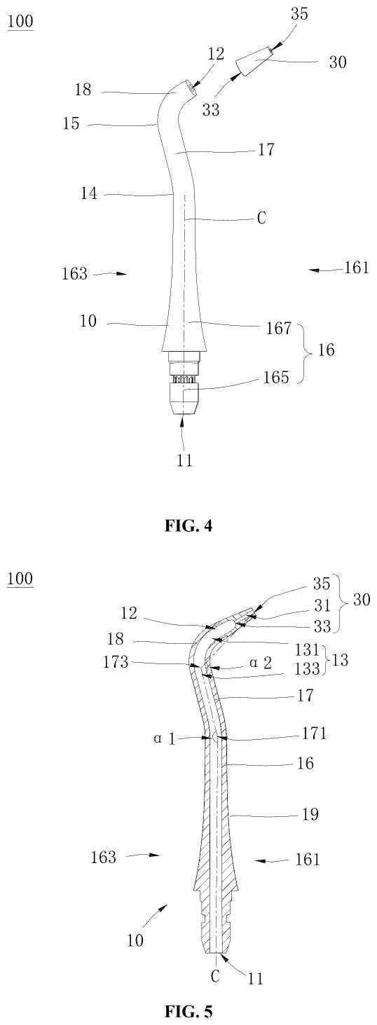

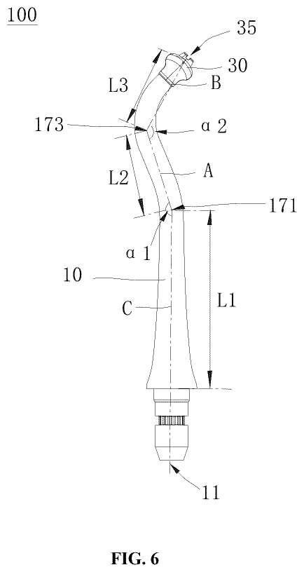

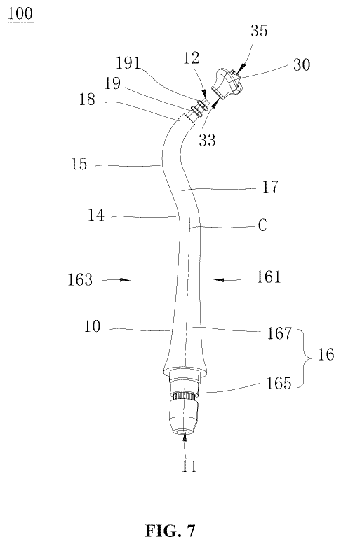

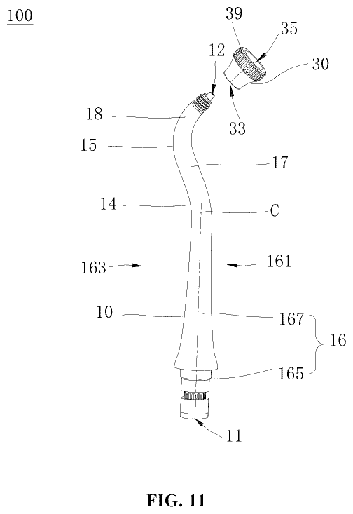

As shown in FIGS. 1-4, 6-7, and 10-11, the nozzle 100 comprises a squirting tube 10. The squirting tube 10 comprises an inlet 11, an outlet 12, and a flow channel 13 communicating the inlet 11 and the outlet 12. The squirting tube 10 at least comprises a first bending position 14 and a second bending position 15, so as to form a main flow section 16, a first bending section 17, and a second bending section 18 in a direction from the inlet 11 to the outlet 12. The main flow section 16, the first bending section 17, and the second bending section 18 are sequentially connected. The first bending section 17 is disposed between the first bending position 14 and the second bending position 15. The second bending section 18 is disposed between the second bending position 15 and the outlet 12. An extension direction of the first bending section 17 is different from an extension direction of the main flow section 16, and the extension direction of the first bending section 17 is different from an extension direction of the second bending section 18. A section of the flow channel 13 in the main flow section 16 defines a first central axis C. The outlet 12 is located on a first side 161 of the first central axis C. The first bending section 17 bends and extends from the main flow section 16 to a second side 163 of the first central axis C. The first side 161 of the first central axis C is opposite to the second side 163 of the first central axis C.

Specifically, the nozzle 100 is configured to squirt a fluid in the main body 300 into an oral cavity of a user in a form of a water floss to clean an area to be cleaned in the oral cavity. The area to be cleaned in the oral cavity may be, but is not limited to areas that are difficult to clean such as gaps between teeth and gingival sulcus. The nozzle 100 may be made from, but is not limited to plastic, silicone, or metal. When the nozzle 100 is made from the plastic, the nozzle 100 is light in weight, low in cost, and easy to process. When the nozzle 100 is made from the silicone, the nozzle 100 is relatively soft, has good elasticity, and will not harm oral tissue. When the nozzle 100 is made from the metal, the nozzle 100 has high strength, long service life, and is easy to clean and maintain.

In some embodiments, the nozzle 100 comprises the squirting tube 10 and a squirting head 30. The squirting tube 10 is configured to connect to the main body 300, and the squirting tube 10 is configured to deliver the fluid in the main body 300 to the squirting head 30. When in use, the fluid in a liquid storage space inside the main body 300 is delivered from the inlet 11 of the squirting tube 10 to the flow channel 13, and then squirted from the outlet 12 to the squirting head 30. The squirting head 30 squirts the fluid to the area to be cleaned in the oral cavity. A shape of a cross section of the flow channel 13 may be, but is not limited to a circle, an ellipse, or a polygon, etc. In one optional embodiment of the present disclosure, the shape of the cross section of the flow channel 13 is the circle. In some embodiments, the squirting tube 10 and the squirting head 30 are an integrated structure, that is, the squirting tube 10 and the squirting head 30 are integrally formed, thereby improving a bonding strength between the squirting tube 10 and the squirting head 30, preventing the squirting tube 10 and the squirting head 30 from separating during an operation of the nozzle 100, and ensuring stability and reliability of the operation of the nozzle 100.

In other embodiments, the squirting tube 10 and the squirting head 30 are separated structures, that is, the squirting tube 10 and the squirting head 30 are two different structures. In one optional embodiment, the squirting tube 10 is detachably connected to the squirting head 30. For instance, the squirting tube 10 is snapped with or screwed with the squirting head 30, which is conducive to the replacement of the squirting head 30 and the squirting tube 10, so that the squirting tube 10 is allowed to be connected to different types of squirting heads. Thus, replacement cost is reduced. In some other optional embodiments, the squirting tube 10 is fixed to the squirting head 30. For instance, the squirting tube 10 and the squirting head 30 are fixed by bonding or welding, etc., so there is no gap at a connection between the squirting tube 10 and the squirting head 30, and the nozzle 100 has a good waterproof effect. The squirting tube 10 and the squirting head 30 may be made form same material. For instance, both of the squirting tube 10 and the squirting head 30 are made from PP plastic. Alternatively, the squirting tube 10 and the squirting head 30 may be made from different materials. For instance, the squirting tube 10 is made from the PP plastic, and the squirting head 30 is made from silicone.

The squirting tube 10 comprises the main flow section 16, the first bending section 17, and the second bending section 18. The squirting tube 10 at least comprises the first bending position 14 and the second bending position 15. A section, from the first bending position 14 to the second bending position 15, of the squirting tube 10, is defined as the first bending section 17. A section, from the second bending position 15 to the outlet 12, of the squirting tube 10, is defined as the second bending section 1. A bending method of the squirting tube 10 is selected from, but is not limited to hot bending, mechanical bending, etc. In one optional embodiment of the present disclosure, the first bending position 14 and the second bending position 15 of the squirting tube 10 are formed by hot bending. The first bending position 14 and the second bending position 15 are formed by hot bending once or twice. It is understood that the flow channel 13 penetrates the main flow section 16, the first bending section 17, and the second bending section 18. A flow area of the section of the flow channel 13 in the main flow section 16, a flow area of a section of the flow channel 13 in the first bending section 17, and a flow area of a section of the flow channel 13 in the second bending section 18 may be the same, different, or partially the same.

Furthermore, the extension direction of the first bending section 17 is different from the extension direction of the main flow section 16. In the embodiment of the present disclosure, the extension direction of the main flow section 16 is in a direction of a Z-axis, and the extension direction of the first bending section 17 is different from the extension direction of the main flow section 16. Namely, the first bending section 17 is bent at the first bending position 14 relative to the main flow section 16. Furthermore, the extension direction of the first bending section 17 is different from the extension direction of the second bending section 18. Namely, the second bending section 18 is bent at the second bending position 15 relative to the first bending section 17. Optionally, the extension direction of the second bending section 18 and the extension direction of the main flow section 16 may be the same or different.

The section of flow channel 13 in the main flow section 16 has the first central axis C. Since the extension direction of the main flow section 16 of the present disclosure is in the direction of the Z axis, and the main flow section 16 is not bent, the first central axis C is a central axis of the main flow section 16. Assuming that a shortest connecting line between a center of the outlet 12 and the first central axis C forms a first plane, with a reference plane perpendicular to the first plane and passing through the first central axis C as a boundary, one side where the outlet 12 is located is the first side 161 of the first central axis C, and the other side where the outlet 12 is not located is the second side 163 of the first central axis C.

Furthermore, the main flow section 16 is a linear column structure. It is understood that the linear column structure refers to a column whose first central axis C is a straight line. The section of the flow channel 13 in the main flow section 16 is also linear, and the flow area of any position of the flow channel 13 in the main flow section 16 is equal, so that when the fluid flows in the main flow section 16, turbulence is not likely to occur, and energy loss of the fluid is small.

When the user uses the oral irrigator 1000, the first bending section 17 and the second bending section 18 reach deeper into the oral cavity of the user than the main flow section 16. The first bending section 17 and the second bending section 18 are bent relative to the main flow section 16, which makes the second bending section 18 being better put into positions such as the gaps between the teeth and gums to be cleaned, of the oral cavity. Further, the first bending section 17 and the second bending section 18 are bent relative to the main flow section 16, taking into account a size of the teeth, the gums and lips compared to the main flow section 16, which is conducive to turning and shifting of the nozzle 100 in the oral cavity. In addition, when using the nozzle 100, the user does not need to significantly adjust a posture of an arm for facilitating comprehensive cleaning of the oral cavity, which further improves the use experience of the user.

The first bending section 17 bends and extends from the main flow section 16 toward the second side 163 of the first central axis C of the main flow section 16, and the second bending section 18 bends and extends from the first bending section 17. When the nozzle 100 is inserted into the oral cavity and cleans a deep part of the oral cavity to be cleaned, the first bending section 17 is closer to the lips and the teeth at a front of the oral cavity than the second bending section 18. The second bending section 18 is deeper into the oral cavity and closer to the teeth at the deep part of the oral cavity than the first bending section 17. In addition, since the first bending section 17 bends and extends toward the second side 163 of the first central axis C of the main flow section 16, the first bending section 17 and the main flow section 16 jointly define a first space on the second side 163 of the first central axis C. The first space provides a space for the lips and the teeth at the front of the oral cavity, so the first bending section 17 does not contact the lips and the teeth at the front of the oral cavity when the nozzle 100 cleans the deep part of the oral cavity to be cleaned. On the one hand, the first space prevents the nozzle 100 from hitting the lips and the teeth at the front of the oral cavity to avoid damage to the human body. On the other hand, the first space increase an amplitude of the nozzle 100 turning and shifting in the oral cavity and expands a cleaning area of the nozzle 100. Moreover, by providing the first bending section 17 and the second bending section 18, the user does not need to open his mouth wide when putting the nozzle 1000 into the mouth.

The main body 300 is configured to provide the fluid and power to the nozzle 100. A pump (not shown) and the liquid storage space are disposed in the main body 300. When the oral irrigator 1000 is started, the pump pumps the fluid in the liquid storage space to the nozzle 100. The fluid stored in the liquid storage space may be clean water, physiological saline, a special flushing fluid, or a liquid with a certain drug component, which is not limited thereto. The main body 300 comprises buttons such as a switch button and a mode selection button. The user is able to choose an appropriate mode according to personal needs to achieve a best cleaning effect. Furthermore, the main body 300 may comprise a display area, which displays information such as power level and a current mode in real time, so that the user is able to obtain a status of the oral irrigator 1000.

In the above embodiment, the nozzle 100 define the first bending position 14 and the second bending position 15, so as to form the first bending section 17 and the second bending section 18, so that the outlet 12 of the nozzle 100 is more retracted. As a result, an effective working area of the nozzle 100 is expanded, the user does not need to open his mouth wide, and it is convenient for the user to insert the nozzle 100 into the oral cavity to clean the residue between the gaps of the teeth deep in the oral cavity, making the cleaning effect good. The outlet 12 is disposed on the first side 161 of the first central axis C. The first bending section 17 bends and extends from the main flow section 16 toward the second side 163 of the first central axis C. When the nozzle 100 is inserted into the oral cavity, the first space formed by the main flow section 16 and the first bending section 17 on the second side 163 of the first central axis C enables the nozzle 100 not to contact various parts of the oral cavity, so that the nozzle 100 is smoothly inserted into the deep part of the oral cavity to clean the teeth.

As shown in FIGS. 2-6 and 10, the first bending section 17 comprises a first connecting port 171 and a second connecting port 173. The first connecting port 171 is communicated with the main flow section 16. The second connecting port 173 is communicated with the second bending section 17. The squirting tube 10 defines a first center connecting line A. The first center connecting line A connects a center of the first connecting port 171 and a center of the second connecting port 173, and an included angle α₁ between the first central axis C and the first center connecting line A is in a range of [150°, 175°].

Specifically, the included angle α₁ may be 150°, 152°, 155°, 157°, 159°, 161°, 164°, 166°, 169°, or 175°. If the included angle α₁ is less than 150°, an deviation angle of the first bending section 17 relative to the first central axis C is too large, causing the second connecting port 173 to deviate too much from the first central axis C. As a result, when the nozzle 100 is inserted into the oral cavity, rotated, and shifted, the second connecting port 173 is likely to touch the teeth or the gums in the oral cavity, which not only hinders the turning and shifting of the nozzle 100 in the oral cavity, but also easily causes damage to the oral cavity. If the included angle α₁ is greater than 175°, the first space defined by the main flow section 16 and the first bending section 17 on the second side 163 of the first central axis C is too small, and the first space is impossible to provide accommodating space for the lips and the teeth at the front of the oral cavity when the nozzle 100 cleans the area to be cleaned deep in the oral cavity, and the nozzle 100 may hit the lips and the teeth at the front of the oral cavity.

When the included angle α₁ between the first central axis C and the first center connecting line A is in the range of [150°, 175°], the nozzle is easily turned and shifted in the oral cavity, and the first space with a proper size is defined. When the nozzle 100 cleans the area to be cleaned deep in the oral cavity, the first space provides the accommodating space for the lips and the teeth at the front of the oral cavity, so the nozzle does not contact the lips and the teeth at the front of the oral cavity.

As shown in FIGS. 2, 4, 7, and 11, the second bending section 18 bends from the first bending section 17 toward the first side 161 of the first central axis C.

Specifically, the second bending section 18 is bent from the first bending section 17 toward the first side 161 of the first central axis C, so that the second bending section 18 is deeper into the oral cavity and is closer to the teeth deep in the oral cavity than the first bending section 17, and the second bending section 18 and the first bending section 17 jointly define a second space on the first side 161 of the first central axis C. When the nozzle 100 cleans the area to be cleaned deep in the oral cavity, the second space provides a space for the teeth or the gums around the area to be cleaned, so the nozzle 100 does not contact the teeth or gums around the area to be cleaned, enabling the second bending section 18 to have a larger turning angle, which is conducive to the nozzle 100 turning and shifting in the oral cavity. Therefore, the fluid squirted by the nozzle 100 is aimed at and clean the areas that are difficult to clean, such as the gaps between the teeth deep in the oral cavity.

Moreover, the second bending position 15 slows down a flow rate of the fluid entering the second bending section 18 from the first bending section 17. The fluid are slowed down for two time by the first bending section 17 and the second bending section 18 respectively, the slow flow of the first bend section 17, which reduces an impact force of the fluid on the oral cavity.

As shown in FIGS. 1, 3, 6, and 10, in some embodiments, the second bending section 18 comprises the second connecting port 173 communicated with the first bending section 17 and the outlet 12. The squirting tube 10 defines a second center connecting line B. The second center connecting line B connects the center of the second connecting port 173 and a center of the outlet 12, and an included angle α₂ between the first center connecting line A and the second center connecting line B is in a range of [90°, 135°].

Specifically, the included angle α₂ may be 90°, 93°, 95°, 99°, 103°, 109°, 114°, 120°, 126°, or 135°. If the included angle α₂ is less than 90°, the second bending section 18 is too downward, and the outlet 12 is too downward accordingly, which reduce the working area. If the included angle α₂ is greater than 135°, the second space defined by the first bending section 17 and the second bending section 18 on the first side 161 of the first central axis C is too small, and an avoidance effect on various parts of the oral cavity is limited.

When the included angle α₂ between the first center connecting line A and the second center connecting line B is in the range of [90°, 135°], the second space with a proper size is defined to avoid various parts of the oral cavity. Further, the included angle α₂ provides a reasonable bending angle, making it easier to clean the teeth deep in the oral cavity.

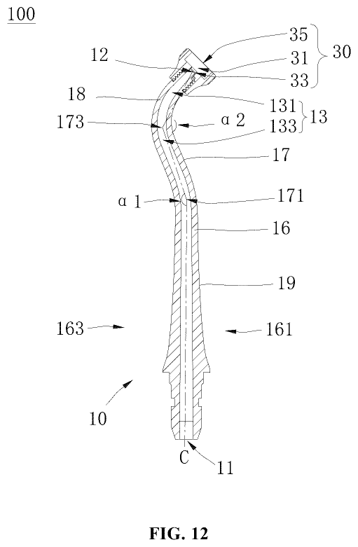

As shown in FIGS. 1-2, 4-5, 7-8, and 11-12, in some embodiments, the squirting head 30 defines a channel 31 penetrating the squirting head 30. The channel 31 is communicated with the flow channel 13. A liquid inlet 33 is defined in a first end of the channel 31 close to the flow channel 13, and a liquid outlet 35 is defined in a second end of the channel 31 away from the flow channel 13. The first bending section 17 comprises the first connecting port 171 and the second connecting port 173. The second bending section 18 comprises the outlet 12. The first connecting port 171 is communicated with the main flow section 16, and the second connecting port 173 is communicated with the second bending section 18. The main flow section 16 comprises a first sub-section 165 and a second sub-section 167. The first sub-section 165 is configured to connect to the main body 300 of the oral irrigator 1000. The second sub-section 167 is connected to the first sub-section 167 and the first bending section 17. The second sub-section 167 has a length L₁, a distance between the first connecting port 171 and the second connecting port 173 is L₂, and a distance between the second connecting port 173 and the liquid outlet 35 is L₃. L₁>L₂>L₃.

Specifically, after the fluid enters the squirting piece 10 from the inlet 11, the fluid flows through the flow channel 13, the outlet 12, the liquid inlet 33, the channel 31, and the liquid outlet 35 in sequence, and finally flows out from the liquid outlet 35 to the area to be cleaned in the oral cavity to clean the oral cavity. It is understood that the outlet 12 is aligned with the liquid inlet 33 to guide the flow of fluid from the flow channel 13 to the channel 31. The flow area of the outlet 12 may be the same as the flow area of the liquid inlet 33, or the flow area of the outlet 12 may be different from the flow area of the liquid inlet 33. The center of the outlet 12 may be the same as a center of the liquid inlet 33, or the center of the outlet 12 may be different from the center of the liquid inlet 33. When the center of the outlet 12 is the same as the center of the liquid inlet 33, the fluid flows smoothly when entering the channel 31 from the flow channel 13. When the center of the outlet 12 is different from the center of the liquid inlet 33, the fluid entering the channel 31 from the outlet 12 have a certain angle with respect to the liquid inlet 33, so that the fluid flowing into the channel 31 impacts the inner wall 37 of the squirting head 30, slowing down the flow rate. The first sub-section 165 is configured to connect to the main body 300 of the oral irrigator 1000. In one embodiment, the first sub-section 165 is detachably connected to the main body 300 to facilitate the maintenance and replacement of the nozzle 100 and/or the main body 300. The first sub-section 165 is detachably connected to the main body 300 by snapping, screwing, etc. In another embodiment, the first sub-section 165 is fixed to the main body 300. Optionally, the first sub-section 165 is fixed to the main body 300 by bonding or welding. In the present disclosure, the first sub-section 165 comprises stepped protrusions and stepped grooves to be detachably connected to the main body 300.

The length L₁ of the second sub-section 167, the distance L₂ between the first connecting port 171 and the second connecting port 173, and the distance L₃ between the second connecting port 173 and the liquid outlet 35 satisfy L₁>L₂>L₃. The length L₁ of the second sub-section 167 is the longest, which enables the nozzle 100 to have an enough length to be inserted deep into the oral cavity. The distance L₃ between the second connecting port 173 and the liquid outlet 35 is the shortest, which is conducive to the turning and shifting of the second bending section 18 in the oral cavity.

As shown in FIGS. 2, 5, 8, and 12, in some embodiments, in a direction from the liquid inlet 33 to the liquid outlet 35, a flow area of the channel 31 gradually increases.

Specifically, the flow area of the channel 31 gradually increases, allowing the fluid to be squirted into the oral cavity in an outwardly expanding manner, thereby expanding a fluid coverage range, and further increasing a cleaning area of the nozzle 100 in the oral cavity. At the same time, it further slows down the flow rate of the fluid in the channel 31 and reduce the flow rate of the fluid at the liquid outlet 35, thereby preventing the impact on the oral cavity due to excessive flow rate of the fluid.

As shown in FIGS. 1, 3, 6, and 10, in some embodiments, a ratio between the length L₁ of the second sub-section 167 and the distance L₂ between the first connecting port 171 and the second connecting port 173 satisfies: L₁:L₂=[1.5, 3].

Specifically, the ratio of L₁:L₂ may be 1.5, 1.8, 2, 2.2, 2.4, 2.6, 2.7, 2.8, 2.9, or 3. If the ratio of L₁:L₂ is less than 1.5, the length L₁ of the second sub-section 167 is insufficient, which is not conducive to the nozzle 100 inserting deep into the oral cavity. If the value of L₁:L₂ is greater than 3, the length L₁ of the second sub-section 167 is too long, resulting in an overall length of the squirting tube 10 being too long, occupying a larger volume, and being not conducive to user operation.

The ratio of L₁:L₂ is [1.5, 3], which makes the length L₁ of the second sub-section 167 to be appropriate, facilitating the nozzle 100 reaching deep into the oral cavity without occupying excessive volume. Additionally, an appropriate ratio of L₁:L₂ is conducive to manufacturing.

As shown in FIGS. 1, 3, 6, and 10, in some embodiments, the ratio between the distance L₂ between the first connecting port 171 and the second connecting port 173 and the distance L₃ between the second connecting port 173 and the liquid outlet 35 satisfies: L₂:L₃=[1.2, 3].

Specifically, the ration of L₂:L₃ may be 1.2, 1.8, 2, 2.2, 2.4, 2.6, 2.7, 2.8, 2.9, or 3. If the ratio of L₂:L₃ is less than 1.2, the distance L₂ between the first connecting port 171 and the second connecting port 173 is relatively close, and the first space defined by the first bending section 17 and the main flow section 16 on the second side 163 of the first central axis C is too small, and the avoidance effect of the first space on various parts of the oral cavity is limited. If the ratio of L₂:L₃ is greater than 3, the distance L₂ between the first connecting port 171 and the second connecting port 173 is relatively far, which is not conducive to the nozzle 100 inserting into the oral cavity.

The ratio of L₂:L₃ is [1.2, 3], so the first space is of reasonable size to provide the accommodating space for the lips and the teeth at the front of the oral cavity when the nozzle 100 cleans the area to be cleaned deep in the mouth, and the nozzle does not contact the lips and the teeth at the front of the oral cavity. At the same time, the ratio of L₂:L₃ is appropriate, which facilitates the nozzle 100 to insert into the oral cavity, and also facilitates turning and shifting of the nozzle 100 in the oral cavity, and is conducive to manufacturing.

As shown in FIGS. 1, 3, 6, and 10, in some embodiments, the first bending section 17 and the main flow section 16 respectively disposed at two sides of the first bending position 14 are smoothly connected to form a first curve. The first bending section 17 and the second bending section 18 on two sides of the second bending position 15 are smoothly connected to form a second curve. A ratio of a bending radius R₁ of the first curve to a bending radius R₂ of the second curve is [1.2, 3.5].

Specifically, the ratio of R₁:R₂ is 1.2, 1.8, 2, 2.2, 2.4, 2.6, 2.7, 2.8, 2.9, or 3.5. If the ratio of R₁:R₂ is less than 1.2, a curvature of the second curve is not enough, causing the nozzle 100 unable to effectively approach the area to be cleaned. Further, when the curvature of the second curve is small, the fluid is not effectively slowed down, the flow rate of the fluid is too large, and the impact force on the oral cavity is too strong, resulting in a poor use experience. If the ratio of R₁:R₂ is greater than 3.5, a bending angle thereof is too large, the first curve is not smooth enough, and dust is easily accumulated at the first bending position 14. Further, if the bending angle is too large, the flow rate of the fluid is not enough to impact on the area to be cleaned, which affects the cleaning effect of the oral cavity.

The ratio of the bending radius R₁ of the first curve to the bending radius R₂ of the second curve is [1.2, 3.5], which ensures that the first curve and the second curve are smooth and is conductive to molding of the nozzle 100. Further, it enables the first bending section 17 and the second bending section 18 to have a buffering effect on the fluid without affecting the impacting force of the fluid on the oral cavity.

As shown in FIGS. 1, 3, 6, and 10, in some embodiments, a longitudinal section of the first bending section 17 is coplanar with a longitudinal section of the second bending section 18.

Specifically, the longitudinal section of the first bending section 17 is a section passing through the first center connecting line A, and the longitudinal section of the second bending section 18 is a section passing through the second center connecting line B. The longitudinal section of the first bending section 17 is coplanar with the longitudinal section of the second bending section 18, so the first connecting port 171, the second connecting port 173, and the outlet 12 are coplanar, which is conductive to molding the nozzle 100 and is convenient for the use to insert the nozzle 100 into the oral cavity.

As shown in FIGS. 1, 3, 6, and 10, in some embodiments, the longitudinal section of the first bending section 17 is not coplanar with the longitudinal section of the second bending section 18.

Specifically, the longitudinal section of the first bending section 17 is not coplanar with the longitudinal section of the second bending section 18, so the outlet 12 is not on the longitudinal section of the first bending section 17, enabling the nozzle 100 to have a larger adjustment angle.

As shown in FIGS. 2, 5, 8, and 12, in some embodiments, the flow channel 13 comprises a first flow channel 131 and a second flow channel 133. The first flow channel 131 is communicated with the second flow channel 133. The main flow section 16 and the first bending section 17 define the first flow channel 131. The second bending section 17 defines the second flow channel 133. A flow area of the first flow channel 131 is greater than a flow area of the second flow channel 133.

Specifically, a minimum flow area of the first flow channel 131 is greater than a maximum flow area of the second flow channel 133, so the fluid flows faster in the second flow channel 133 after passing through the first flow channel 131, thereby increasing a water pressure of the fluid squirted from the outlet 12 and improving the cleaning effect.

It should be noted that the squirting tube 10 is selected from squirting tubes 10 of different specifications. The squirting head 30 is selected from squirting heads 30 of different specifications. Squirting tubes 10 and squirting heads 30 of different specifications are disposed and combined to form nozzles 100 of different specifications. For instance, the squirting tubes 10 of different specifications may be connected to squirting heads 30 of same specification to form different nozzles 100, or the squirting tubes 10 of different specification may be connected to squirting heads 30 of different specifications to form different nozzles 100, or the squirting heads 30 of different specifications are connected to the squirting tubes 10 of different specifications to form the nozzles 100 of different specifications.

For instance, the following embodiments disclose a same squirting tube 10 connected to two different squirting heads 30 to form different nozzles 100. The squirting tube 10 connected to the two different squirting heads 30 is the squirting tube 10 in the above embodiments, and at least has the effects of the above squirting tube 10.

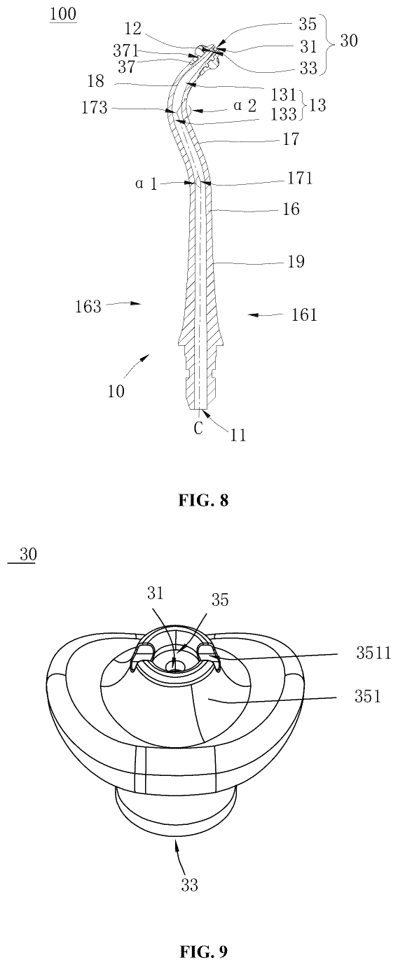

As shown in FIGS. 7 and 8, grooves 371 are defined in an inner wall of the squirting head 30. Limiting protrusions 191 are disposed on an outer wall of the squirting tube 10. The limiting protrusions 191 are respectively engaged with the grooves 371, so that the squirting tube 10 is connected to the squirting head 30. That is, the squirting tube 10 and the squirting head 30 are connected to each other through cooperation between the grooves 371 and the limiting protrusions 191, so that the squirting head 30 is not easy to fall off.

As shown in FIG. 8, in some embodiments, the squirting head 30 is made from the soft rubber.

Specifically, the squirting head 30 is made from the soft rubber such as silicone, latex, or synthetic rubber. The squirting head 30 made from the soft rubber has good elasticity and flexibility, which fits the area to be cleaned more closely, prevents the squirting head 30 from hitting the gums or the teeth, and prevents the squirting head 30 from damaging the oral cavity.

As shown in FIG. 9, in some embodiments, a peripheral wall 351 of the liquid outlet 35 defines notches 3511.

Specifically, the notches 3511 are defined on the peripheral wall 351 of the liquid outlet 35 and do not extend to the liquid inlet 33, which ensure that a structural strength of the squirting head 30 is sufficient and is not prone to vibration nor cracking after long-term use. When the squirting head 30 is made from a soft material, the notches 3511 enables the squirting head 30 to have a certain distance from the squirting tube 10 after deformation, so that the squirting tube 10 does not directly contact the teeth. In addition, when cleaning any gap between teeth, one of the notches 3511 abuts against edges of two teeth defining the gap, so that the liquid outlet 35 fits the teeth while reserving a certain space for the fluid to squirt outward, thereby expanding a flushing area of the nozzle 100. The notches 3511 also make the squirting head 30 more likely to deform, so as to adapt to the gaps, of different depths, between the teeth, so the nozzle is able to better clean the gaps between teeth.

As shown in FIG. 8, in some embodiments, a width of an outer contour of a longitudinal section of the squirting head 30 is gradually increased and then gradually reduced in the direction from the liquid inlet 33 to the liquid outlet 35,

Specifically, when cleaning any gap between teeth, a maximum width of the outer contour of the longitudinal section of the squirting head 30 abuts against tooth surfaces of two adjacent teeth defining the gap, so that the liquid outlet 35 squirt water toward the gap between the two adjacent teeth to clean the gap. The width of the outer contour of the longitudinal section of the squirting head 30 gradually increases and then gradually decreases. That is, a width of the liquid outlet 35 is small, which allows the liquid outlet 35 to extend into a smaller area in the oral cavity and clean the small area, which facilitates turning and shifting of the liquid outlet 35 in the smaller area.

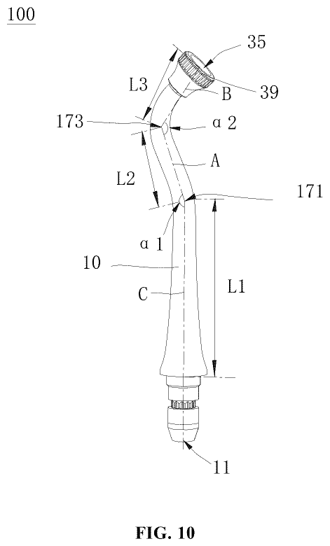

As shown in FIG. 10, in some embodiments, the outer wall 19 of the squirting tube 10 is screwed with the inner wall 37 of the squirting head 30 by threads, which facilitates the disassembly and replacement of the squirting head 30. Bristles 39 are disposed on one end of the squirting head 30 away from the squirting tube 10. Namely, the bristles 39 are disposed around the liquid outlet 35. The bristles 39 improves the cleaning effect. When the oral irrigator flushes the area to be cleaned, the bristles 39 sweep away the residues on a surface of the area to be cleaned. The squirting head 30 is made from a hard material or the soft material. For instance, the squirting head 30 is made from the hard material, such as polypropylene (PP). The squirting head 30 made from the hard material has good corrosion resistance and high temperature resistance, and is allowed to be used for a long time in different environments. The bristles 39 may be made from the same material as the squirting head 30, or the bristles 39 and the squirting head are made from different materials.

What is claimed is:

1. A nozzle of an oral irrigator, comprising: a squirting tube;
wherein the squirting tube comprises an inlet, an outlet, and a flow channel communicating the inlet and the outlet;
wherein the squirting tube at least comprises a first bending position and a second bending position, so as to form a main flow section, a first bending section; and a second bending section in a direction from the inlet to the outlet, the main flow section, the first bending section, and the second bending section are sequentially connected, the first bending section is disposed between the first bending position and the second bending position, the second bending section is disposed between the second bending position and the outlet, an extension direction of the first bending section is different from an extension direction of the main flow section, and the extension direction of the first bending section is different from an extension direction of the second bending section;
wherein a section of the flow channel in the main flow section defines a first central axis, the outlet is located on a first side of the first central axis, the first bending section bends and extends from the main flow section to a second side of the first central axis, and the first side of the first central axis is opposite to the second side of the first central axis.

2. The nozzle according to claim 1, wherein the first bending section comprises a first connecting port and a second connecting port, the first connecting port is communicated with the main flow section, the second connecting port is communicated with the second bending section, the squirting tube defines a first center connecting line, the first center connecting line connects a center of the first connecting port and a center of the second connecting port, and an included angle α₁ between the first central axis and the first center connecting line is [150°, 175°].

3. The nozzle according to claim 1, wherein the second bending section bends from the first bending section toward the first side of the first central axis.

4. The nozzle according to claim 3, wherein the first bending section comprises a first connecting port, the second bending section comprises the outlet, the first bending section and the second bending section shares a second connecting port, the first bending section is communicated with the second bending section through the second connecting port, the first connecting port is communicated with the main flow section, the squirting tube defines a first center connecting line, the first center connecting line connects a center of the first connecting port and a center of the second connecting port, the squirting tube defines a second center connecting line, the second center connecting line connects the center of the second connecting port and a center of the outlet, and an included angle α₂ between the first center connecting line and the second center connecting line is [90°, 135°].

5. The nozzle according to claim 1, wherein the nozzle further comprises a squirting head connected to the squirting tube, the squirting head defines a channel penetrating the squirting head from a first end of the squirting head to a second end of the squirting head, and the channel is communicated with the flow channel;
wherein a liquid inlet is defined in a first end of the channel close to the flow channel, and a liquid outlet is defined in a second end of the channel away from the flow channel;
wherein the first bending section comprises a first connecting port, the second bending section comprises the outlet, the first bending section and the second bending section shares a second connecting port, the first bending section is communicated with the second bending section through the second connecting port, and the first connecting port is communicated with the main flow section;
wherein the main flow section comprises a first sub-section and a second sub-section, the first sub-section is configured to connect to a main body of the oral irrigator, the second sub-section is connected to the first sub-section and the first bending section, the second sub-section has a length L₁, a distance between the first connecting port and the second connecting port is L₂, a distance between the second connecting port and the liquid outlet is L₃, and L₁>L₂>L₃.

6. The nozzle according to claim 5, wherein in a direction from the liquid inlet to the liquid outlet, a flow area of the channel gradually increases.

7. The nozzle according to claim 5, wherein L₁:L₂ is in a range of [1.5, 3]; and/or
L₂:L₃ is in a range of [1.2, 3].

8. The nozzle according to claim 1, wherein the first bending section and the main flow section respectively disposed at two sides of the first bending position are smoothly connected to form a first curve, the first bending section and the second bending section on two sides of the second bending position are smoothly connected to form a second curve, a ratio of a bending radius R₁ of the first curve to a bending radius R₂ of the second curve is [1.2, 3.5].

9. The nozzle according to claim 1, wherein a longitudinal section of the first bending section is coplanar with a longitudinal section of the second bending section; or
the longitudinal section of the first bending section is not coplanar with the longitudinal section of the second bending section.

10. The nozzle according to claim 1, wherein the flow channel comprises a first flow channel and a second flow channel, the first flow channel is communicated with the second flow channel, the main flow section and the first bending section define the first flow channel, the second bending section defines the second flow channel, and a flow area of the first flow channel is greater than a flow area of the second flow channel.

11. The nozzle according to claim 1, wherein the nozzle further comprises a squirting head connected to the squirting tube;
wherein the squirting tube and the squirting head are of an integrated structure; or
the squirting tube and the squirting head are separate structures, and the squirting tube is detachably connected to the squirting head; or
the squirting tube and the squirting head are separate structures, and the squirting tube is fixedly connected to the squirting head.

12. The nozzle according to claim 11, wherein at least one groove is defined in an inner wall of the squirting head, at least one limiting protrusion is disposed on an outer wall of the squirting tube, and the at least one limiting protrusion is engaged with the at least one groove, so that the squirting tube is connected to the squirting head.

13. The nozzle according to claim 12, wherein the squirting head is made from soft rubber.

14. The nozzle according to claim 12, wherein the squirting head defines a channel, the channel penetrates the squirting head from a first end of the squirting head to a second end of the squirting head, the channel is communicated with the flow channel, a liquid inlet is defined in a first end of the channel close to the flow channel, a liquid outlet is defined in a second end of the squirting head away from the flow channel, and a peripheral wall of the liquid outlet defines at least one notch.

15. The nozzle according to claim 12, wherein the squirting head defines a channel, the channel penetrates the squirting head from a first end of the squirting head to a second end of the squirting head, the channel is communicated with the flow channel, a liquid inlet is defined in a first end of the channel close to the flow channel, a liquid outlet is defined in a second end of the squirting head away from the flow channel, and a width of an outer contour of a longitudinal section of the squirting head is gradually increased and then gradually reduced.

16. The nozzle according to claim 11, wherein an outer wall of the squirting tube is screwed with an inner wall of the squirting head, and bristles are disposed on a first end of the squirting head away from the squirting tube.

17. An oral irrigator, comprising:
a nozzle; and
a main body;
wherein the nozzle comprises a squirting tube; the squirting tube comprises an inlet, an outlet, and a flow channel communicating the inlet and the outlet;
wherein the squirting tube at least comprises a first bending position and a second bending position, so as to form a main flow section, a first bending section, and a second bending section in a direction from the inlet to the outlet, the main flow section, the first bending section, and the second bending section are sequentially connected; the first bending section is disposed between the first bending position and the second bending position, the second bending section is disposed between the second bending position and the outlet, an extension direction of the first bending section is different from an extension direction of the main flow section, and the extension direction of the first bending section is different from an extension direction of the second bending section;
wherein a section of the flow channel in the main flow section defines a first central axis, the outlet is located on a first side of the first central axis, the first bending section bends and extends from the main flow section to a second side of the first central axis, and the first side of the first central axis is opposite to the second side of the first central axis;
wherein the main body is connected to the nozzle.

18. The oral irrigator according to claim 17, wherein the first bending section comprises a first connecting port and a second connecting port, the first connecting port is communicated with the main flow section, the second connecting port is communicated with the second bending section; the squirting tube defines a first center connecting line, the first center connecting line connects a center of the first connecting port and a center of the second connecting port, and an included angle α₁ between the first central axis and the first center connecting line is [150°, 175°]; and/or
the first bending section comprises the first connecting port, the second bending section comprises the outlet, the first bending section and the second bending section shares the second connecting port, the first bending section is communicated with the second bending section through the second connecting port, the squirting tube defines the first center connecting line and a second center connecting line, the first center connecting line connects the center of the first connecting port and the center of the second connecting port, the second center connecting line connects the center of the second connecting port and a center of the outlet, and an included angle α₂ between the first center connecting line and the second center connecting line is [90°, 135°].

19. The oral irrigator according to claim 17, wherein the nozzle further comprises a squirting head connected to the squirting tube, the squirting head defines a channel penetrating the squirting head from a first end of the squirting head to a second end of the squirting head, and the channel is communicated with the flow channel;
wherein a liquid inlet is defined in a first end of the channel close to the flow channel, and a liquid outlet is defined in a second end of the channel away from the flow channel;
wherein the first bending section comprises a first connecting port, the second bending section comprises the outlet, the first bending section and the second bending section share a second connecting port, the first connecting port is communicated with the main flow section, and the second bending section is communicated with the first bending section through the second connecting port;
wherein the main flow section comprises a first sub-section and a second sub-section, the first sub-section is configured to connect to the main body of the oral irrigator, the second sub-section is connected to the first sub-section and the first bending section, the second sub-section has a length L₁, a distance between the first connecting port and the second connecting port is L₂, a distance between the second connecting port and the liquid outlet is L₃, and L₁>L₂>L₃.

20. The oral irrigator according to claim 17, wherein the first bending section and the main flow section respectively disposed at two sides of the first bending position are smoothly connected to form a first curve, the first bending section and the second bending section respectively disposed on two sides of the second bending position are smoothly connected to form a second curve, a ratio of a bending radius R₁ of the first curve to a bending radius R₂ of the second curve is [1.2, 3.5].
